(19) United States
(12) Patent Application Publication
(10) Pub. No.: US 2024/0117052 A1
(43) Pub. Date: Apr. 11, 2024

(54) ANTIBODY FOR CANCER TREATMENT CONJUGATED TO TUMOR ENVIRONMENT-SENSITIVE TRACELESS-CLEAVABLE POLYETHYLENE GLYCOL AND MANUFACTURING METHOD THEREOF

(71) Applicant: RESEARCH & BUSINESS FOUNDATION SUNGKYUNKWAN UNIVERSITY, Suwon-si (KR)

(72) Inventors: Jae Hyung PARK, Suwon-si (KR); Seok Ho SONG, Suwon-si (KR); Torsha GHOSH, Suwon-si (KR)

(73) Assignee: RESEARCH & BUSINESS FOUNDATION SUNGKYUNKWAN UNIVERSITY, Suwon-si (KR)

(21) Appl. No.: 18/376,920

(22) Filed: Oct. 5, 2023

(30) Foreign Application Priority Data
Oct. 5, 2022 (KR) 10-2022-0127426

(51) Int. Cl. C07K 16/28; A61K 47/10; A61P 35/00

(52) U.S. Cl. CPC C07K 16/2818; A61K 47/10; A61P 35/00

(57) ABSTRACT

Disclosed is an antibody for cancer treatment conjugated with a tumor environment-sensitive cleavable polyethylene glycol, wherein the use of the antibody for cancer treatment can suppress side effects of the antibody while maintaining therapeutic effects thereof.

### CROSS-REFERENCE TO RELATED APPLICATION(S)

This application is based on and claims priority under 35 U.S.C. 119 to Korean Patent Application No. 10-2022-0127426, filed on Oct. 5, 2022, in the Korean Intellectual Property Office, the disclosure of which is herein incorporated by reference in its entirety.

### BACKGROUND OF THE INVENTION

### 1. Field of the Invention

The present disclosure was established with the support of the Ministry of Science and ICT of the Republic of Korea under Project No. 2021R1A4A1032782, conducted by Sungkyunkwan University Industry-Academic Cooperation Foundation in the research project entitled “Development of Stem Cell-Derived Artificial Exosomes for Ischemic Disease Treatments” within the research program named “Basic Research Laboratory Support Program” under the management of the National Research Foundation of Korea, from 1 Jun. 2021 to 29 Feb. 2024.

The present disclosure was established with the support of the Ministry of Science and ICT of the Republic of Korea under Project No. 2018R1A2B3006080, conducted by Sungkyunkwan University Industry-Academic Cooperation Foundation in the research project entitled “Development of Multiplatform Materials for Microenvironment-Recognizable Image-Guided Sonodynamic Therapy of Cancer” within the research program named “Mid-Career Research (total research budget exceeding 500 million won)” under the management of the National Research Foundation of Korea, from 1 Mar. 2018 to 28 Feb. 2023.

The present disclosure was established with the support of the Ministry of Health and Welfare, Republic of Korea, under Project No. HN22C0624000022, conducted by Sungkyunkwan University Industry-Academic Cooperation Foundation in the project named “Development of Tumor-Derived Exosome Rupture for Combinational Immunotherapy of Colorectal Cancer” within the research program titled “National New Drug Development—New Drug Base Expansion Research” under the management of the Korea Health Industry Development Institute, from 1 Jun. 2022 to 31 May 2025.

This application claims priority and the benefit of Korean Patent Application No. 10-2022-0127426 filed in the Korean Intellectual Property Office on 5 Oct. 2022, the disclosure of which is incorporated herein by reference.

The present disclosure relates to an antibody for cancer treatment conjugated to tumor environment-sensitive cleavable polyethylene glycol and a manufacturing method thereof.

### 2. Description of the Prior Art

Several antibodies for cancer treatment are being used for cancer therapy. Of these, immune checkpoint inhibitor antibodies were effective in several types of tumors and achieved significant advances in cancer therapies. However, these therapies caused problems called immune-related side effects. It was reported that in actual clinical trials, the administration of immune checkpoint inhibitor antibodies alone caused immune-related side effects in 20-60% of patients whereas the co-administration with other drugs increased the proportion to 90% or more. Moreover, it was known that such antibodies not only cause problems that simply require the reduction of doses or the stopping of treatment but also can endanger life or result in permanent organ damage in worst cases. These side effects of immune checkpoint inhibitor antibodies are currently being addressed by managing the immune response using glucocorticoids, but some patients require life-long treatment, such as hormone supplementation or immunosuppression, due to chronic side effects, and ultimately has reduced life quality without enjoying therapeutic effects of immune checkpoint inhibitor antibodies.

Therefore, there is a need for specific strategies that can alleviate immune-related side effects without compromising the doses and therapeutic effects of antibodies for cancer treatment.

### SUMMARY OF THE INVENTION

The present inventors have made extensive research efforts to develop antibodies for cancer treatment that prevent immune-related side effects and maximize therapeutic efficacy even without the adjustment of drug doses or administration of additional drugs. As a result, the present inventors established that the conjugation of tumor environment-sensitive cleavable polyethylene glycol to antibodies for cancer treatment can suppress immune-related side effects of the antibodies for cancer treatment while maintaining the therapeutic effects thereof, and thus completed the present disclosure.

Accordingly, an aspect of the present disclosure is to provide a particle including: an antibody for cancer treatment; and tumor environment-sensitive cleavable polyethylene glycol (PEG).

Another aspect of the present disclosure is to provide an anticancer pharmaceutical composition containing a particle, the particle including: an antibody for cancer treatment; and tumor environment-sensitive cleavable polyethylene glycol (PEG).

In accordance with an aspect of the present disclosure, there is provided a particle including: an antibody for cancer treatment; and tumor environment-sensitive cleavable polyethylene glycol (PEG).

The present inventors have made extensive research efforts to develop antibodies for cancer treatment that prevent immune-related side effects and maximize therapeutic efficacy even without the adjustment of drug doses or administration of additional drugs. As a result, the present inventors established that the conjugation of tumor environment-sensitive cleavable polyethylene glycol to antibodies for cancer treatment can suppress immune-related side effects of the antibodies for cancer treatment while maintaining the therapeutic effects thereof.

As used herein, the term “polyethylene glycol (PEG)” refers to a type of polyether compound, which is a chemically and biologically actively used substance. In particular, the polyethylene glycol may be used for the protection of an active ingredient. For example, protein drugs, when orally administered, may show reduced medicinal efficacy due to the breakage of active ingredients caused by proteases in the digestive system. Even if injected directly into a vein or subcutaneous fat, the protein drugs may be filtered by the kidneys to result in a fast reduction in the in-vivo concentrations thereof and may cause unnecessary immune responses due to immunogenicity thereof. Therefore, the conjugation of polyethylene glycol derivatives to proteins as active ingredients can prevent the binding of proteases to proteins, reduce the filtration of proteins in the kidneys due to increased molecular weights thereof, and reduce the immunogenicity of proteins by blocking epitopes of the proteins. This is called PEGylation.

As used herein, the term “tumor environment” refers to the environmental totality where cancer cells proliferate and evolve, the environment including fibroblasts, blood vessels and lymphatic vessels, immune cells, extracellular matrix, and adipocytes present within the cancer tissue, besides cancer cells. The components of the tumor microenvironment greatly affect cancer progression, cancer cell metastasis, and therapeutic response and effectiveness through various interactions. The tumor microenvironment is characterized by low oxygen, high expression of specific proteins and enzymes, and a slightly acidic condition (pH 5.5 to 6.5). Herein, the “tumor environment” may preferably mean a slightly acidic condition (pH 5.5 to 6.5). As used herein, the term “tumor environment-sensitive cleavable polyethylene glycol” refers to the cleavage of the conjugation of an antibody for cancer treatment and cleavable polyethylene glycol in a tumor environment. Preferably, the tumor environment may mean a slightly acidic condition (pH 5.5 to 6.5), wherein the tumor environment-sensitive cleavable polyethylene glycol may mean slightly acidic pH-sensitive cleavable polyethylene glycol in the tumor environment.

In an embodiment of the present disclosure, the tumor environment-sensitive cleavable polyethylene glycol is a linkage of a pH-sensitive molecule and polyethylene glycol.

In an embodiment of the present disclosure, the pH-sensitive molecule constituting the tumor environment-sensitive cleavable polyethylene glycol is an acid labile linker or a dimethyl maleic anhydride derivative.

As used herein, the term “acid labile linker” refers to a pH-sensitive compound that is hydrolyzed at a specific acidic pH value. Examples of the pH-sensitive compound may include a hydrazone, semicarbazone, thiosemicarbazone, cis-aconitic amide, orthoester, acetal, ketal, or the like, but are not limited thereto.

In an embodiment of the present disclosure, the dimethyl maleic anhydride derivative is carboxylated dimethyl maleic anhydride (CDM).

In an embodiment of the present disclosure, the tumor environment-sensitive cleavable polyethylene glycol may be prepared by stirring, for example, chlorinated carboxy dimethylmaleic anhydride (CDM) in the presence of polyethylene glycol and a pyridine catalyst. The chlorinated CDM may be prepared by stirring CDM and oxalyl chloride in the presence of a dimethyl formamide catalyst. The stirring may be conducted multiple times, and the temperature for first stirring may be lower than the temperature for second stirring. For example, the first stirring may be performed at −5° C. to 5° C., and the second stirring may be performed at room temperature.

The tumor environment-sensitive cleavable polyethylene glycol containing the pH-sensitive molecule CDM (PEG-CDM) showed a peak at 3.5 to 3.8 ppm(δ), a peak at 3.2 to 3.5 ppm(δ), and a peak at 2.0 to 2.5 ppm(δ), as a result of ¹H-NMR measurement.

In an embodiment of the present disclosure, the tumor environment-sensitive cleavable polyethylene glycol is conjugated to an antibody for cancer treatment.

The tumor environment-sensitive cleavable polyethylene glycol-conjugated antibody for cancer treatment may be manufactured by adding tumor environment-sensitive cleavable polyethylene glycol to an antibody for cancer treatment, followed by stirring. The antibody for cancer treatment may undergo a first buffer treatment step before the addition of the cleavable polyethylene glycol and may undergo a second buffer treatment step after the addition of the cleavable polyethylene glycol. The first buffer may be more basic than the second buffer. For example, the first buffer may be a buffer of pH 8.5 to 9.5, and the second buffer may be a buffer of pH 7.0 to 8.0.

In an embodiment of the present disclosure, the tumor environment-sensitive cleavable polyethylene glycol is bound to an amino group on the surface of the antibody for cancer treatment.

In an embodiment of the present disclosure, the binding may be covalent binding. For example, the binding may be covalent binding with an amino group of an antibody. However, the binding is not limited thereto, and may be covalent binding with any functional group that can form a covalent bond with cleavable polyethylene glycol.

In an embodiment of the present disclosure, the conjugation of the antibody for cancer treatment and the tumor environment-sensitive cleavable polyethylene glycol is dissociated at pH 5.0 to 7.0.

The cleavable polyethylene glycol sensitive to the slightly acidic environment of tumor may be referred to as “pH-sensitive cleavable polyethylene glycol”, and as used herein, the term “pH-sensitive cleavable” refers to being cleavable of the conjugation of polyethylene glycol and an antibody for cancer treatment under a specific pH condition. The specific pH condition may be, for example, pH 5.0 to 7.0, pH 5.0 to 6.9, pH 5.0 to 6.8, pH 5.0 to 6.7, pH 5.0 to 6.6, pH 5.0 to 6.5, pH 5.0 to 6.4, pH 5.0 to 6.3, pH 5.0 to 6.2, pH 5.0 to 6.1, pH 5.0 to 6.0, pH 5.0 to 5.9, pH 5.0 to 5.8, pH 5.0 to 5.7, pH 5.0 to 5.6, pH 5.0 to 5.5, pH 5.0 to 5.4, pH 5.0 to 5.3, pH 5.0 to 5.2, pH 5.0 to 5.1, pH 5.1 to 7.0, pH 5.2 to 7.0, pH 5.3 to 7.0, pH 5.4 to 7.0, pH 5.5 to 7.0, pH 5.6 to 7.0, pH 5.7 to 7.0, pH 5.8 to 7.0, pH 5.9 to 7.0, pH 6.0 to 7.0, pH 6.1 to 7.0, pH 6.2 to 7.0, pH 6.3 to 7.0, pH 6.4 to 7.0, pH 6.5 to 7.0, pH 6.6 to 7.0, pH 6.7 to 7.0, pH 6.8 to 7.0, pH 6.9 to 7.0, pH 5.1 to 6.9, pH 5.2 to 6.8, pH 5.3 to 6.7, pH 5.4 to 6.7, pH 5.5 to 6.7, pH 5.6 to 6.7, pH 5.7 to 6.7, pH 5.8 to 6.7, pH 5.9 to 6.7, pH 6.0 to 6.7, pH 6.1 to 6.7, pH 6.2 to 6.7, or pH 6.3 to 6.7.

In an embodiment of the present disclosure, the antibody for cancer treatment is an immune checkpoint inhibitor (ICI) antibody.

As used herein, the term “immune checkpoint” refers to a receptor that negatively regulates the proliferation or function of T cells in charge of a central role in adaptive immunity. Examples of the receptor include cytotoxic T-lymphocyte associated protein 4 (CTLA4), programmed cell death protein 1 (PD-1), lymphocyte-activation gene-3 (LAG3), killer cell immunoglobulin-like receptor (KIR), tumor necrosis factor receptor superfamily 9 (4-1BB/TNFRS9), T-cell immunoglobulin and mucin-domain containing-3 (TIM3), and the like. Tumor cells can perform immune suppression or evasion through a mechanism where a ligand for an immune checkpoint, such as PD-L1/2, is expressed.

As used herein, the term “immune checkpoint inhibitor antibody” or “immune checkpoint inhibitor” refers to a drug that prevents the immune suppression or evasion of tumor cells by suppressing an immune checkpoint.

In an embodiment of the present disclosure, the “immune checkpoint inhibitor antibody” or “immune checkpoint inhibitor” is an antibody or an antigen-binding fragment thereof that targets at least one selected from the group consisting of CD47, PD-1, PD-L1, CTLA-4, B7-1, B7-2, LAG3, KIR, 4-1BB, and TIM-3.

As used herein, the term “antibody” includes not only a complete antibody form but also an antigen-binding fragment of the antibody molecule. Examples of the antibody include monoclonal antibodies, multi-specific antibodies, human antibodies, humanized antibodies, chimeric antibodies, single-chain Fvs (scFVs), single-chain antibodies, Fab fragments, F(ab′) fragments, disulfide-linked Fvs (sdFVs), anti-idiotype (anti-Id) antibodies, and epitope-binding fragments of the above-mentioned antibodies, but are not limited thereto.

The complete antibody has a structure having two full-length light chains and two full-length heavy chains, wherein the light chains are linked to the heavy chains via disulfide linkages, respectively. Heavy chain constant domains have gamma (γ), mu (μ), alpha (α), delta (δ) and epsilon (ε) types, which are sub-classed into gamma 1 (γ1), gamma 2 (γ2), gamma 3 (γ3), gamma 4 (γ4), alpha 1 (α1) and alpha 2 (α2). Light chain constant domains have kappa (κ) and lambda (λ) types (Cellular and Molecular Immunology, Wonsiewicz, M. J., Ed., Chapter 45, pp. 41-50, W. B. Saunders Co. Philadelphia, P A (1991); and Nisonoff, A., Introduction to Molecular Immunology, 2^{nd} Ed., Chapter 4, pp. 45-65, sinauer Associates, Inc., Sunderland, MA (1984)).

As used herein, the term “antigen-binding fragment” refers to a fragment that retains an antigen-binding function, and examples thereof include Fab, F(ab′), F(ab′)2, Fv, and the like. Out of the antibody fragments, Fab has a structure having light chain and heavy chain variable domains, a light chain constant domain, and the first heavy chain constant domain (CH1), with one antigen-binding site. Fab′ differs from Fab in that the former has a hinge region containing at least one cysteine residue at the C-terminus of the heavy chain CH1 domain. F(ab′)₂ antibody is generated by a disulfide bond formed between cysteine residues of the high regions of Fab′ fragments. Fv is a minimal antibody segment having only a heavy chain variable domain and a light chain variable domain, and recombinant techniques for producing Fv fragments are disclosed in WO 88/10649, WO 88/106630, WO 88/07085, WO 88/07086, and WO 88/09344. Two-chain Fv is a fragment in which a heavy chain variable domain and a light chain variable domain are linked by a non-covalent bond, and single-chain Fv is a fragment in which a heavy chain variable domain and a light chain variable domain are generally linked by a covalent bond via a peptide linker or are directly linked at the C-terminal, forming a dimer-like structure, like the two-chain Fv. These antibody fragments may be obtained using proteolytic enzymes (e.g., the Fab fragment can be obtained by restriction-cleaving the whole antibody with papain and the F(ab′)₂ fragment can be obtained by restriction-cleaving the whole antibody with pepsin), or may be fabricated by genetic recombinant techniques.

In the present disclosure, the antibody is preferably a Fab form or a complete antibody form. The heavy chain constant domains may be any one isotype selected from gamma (γ), mu (μ), alpha (α), delta (δ), or epsilon (ε) type. The light chain constant domains may be kappa or lambda type.

In a specific embodiment of the present disclosure, the proportion of amino groups bound to cleavable polyethylene glycol among the amino groups on the surface of the antibody is 1% to 80%. The proportion of amino groups bound to cleavable polyethylene glycol among the amino groups on the surface of the antibody is not limited thereto, and may be for example 1% to 80%, 1% to 70%, 1% to 60%, 1% to 50%, 1% to 40%, 1% to 30%, 1% to 20%, 1% to 10%, 10% to 80%, 20% to 80%, 30% to 80%, 40% to 80%, 50% to 80%, 60% to 80%, 70% to 80%, 10% to 70%, 20% to 60%, or 30% to 50%.

In an embodiment of the present disclosure, the size of the particle is 10 nm to 200 nm in diameter. The size of the particle is not limited thereto, and may be for example, 10 nm to 150 nm, 10 nm to 100 nm, 10 nm to 50 nm, 10 nm to 45 nm, 10 nm to 40 nm, 10 nm to 35 nm, 10 nm to 30 nm, 10 nm 10 to 25 nm, 10 nm to 20 nm, 10 nm to 15 nm, 20 nm to 200 nm, 30 nm to 200 nm, 40 nm to 200 nm, 50 nm to 200 nm, 60 nm to 200 nm, 70 nm to 200 nm, 80 nm to 200 nm, 90 nm to 200 nm, 100 nm to 200 nm, 150 nm to 200 nm, 20 nm to 25 nm, 25 nm to 50 nm, 30 nm to 50 nm, 35 nm to 50 nm, 40 nm to 50 nm, 45 nm to 50 nm, 25 nm to 45 nm, 30 nm to 45 nm, 30 nm to 40 nm, or 30 nm to 35 nm.

In an aspect of the present disclosure, there is provided an anticancer pharmaceutical composition containing a particle, the particle including: an antibody for cancer treatment; and tumor environment-sensitive cleavable polyethylene glycol.

As used herein, the term “anticancer” refers to the treatment or prevention of a cancer disease.

The anticancer composition of the present disclosure may contain a pharmaceutically acceptable carrier. The pharmaceutically acceptable carrier is typically used at the time of formulation, and examples thereof may include, but are not limited to, lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methylhydroxybenzoate, propylhydroxybenzoate, talc, magnesium stearate, mineral oils, and the like. The pharmaceutical composition of the present disclosure may further contain, in addition to the above ingredients, a lubricant, a wetting agent, a sweetening agent, a flavoring agent, an emulsifier, a suspending agent, a preservative, and the like, but is not limited thereto. Appropriate pharmaceutically acceptable carriers and agents are described in detail in Remington's Pharmaceutical Sciences (19th ed., 1995).

The pharmaceutical composition of the present disclosure may be formulated by using a pharmaceutically acceptable carrier and/or excipient according to a method that can be easily performed by a person skilled in the art to which the present disclosure pertains, and thus the pharmaceutical composition may be provided as a unit dosage form or may be packed in a multi-dose container. In particular, the formulation may be in the form of a solution in an oily or aqueous medium, a suspension, an emulsion, an extract, a pulvis, a suppository, a powder, granules, a tablet, or a capsule, and may further contain a dispersant or a stabilizer.

In an embodiment of the present disclosure, the composition selectively exhibits an anticancer effect in the tumor microenvironment of pH 5.0 to 7.0.

As used herein, the term “selectively” refers to exhibiting the effect of an active ingredient under specific conditions or situations. For example, the meaning that an anti-cancer effect is selectively exhibited in the tumor microenvironment may be that an active ingredient specifically binds to a biomarker for a component constituting the tumor microenvironment, and may be that an active ingredient is activated under low-oxygen conditions in the tumor microenvironment. Alternatively, the meaning may be that an active ingredient is activated under a slightly acidic condition of the tumor microenvironment, but is not limited thereto. The meaning that an anti-cancer effect is selectively exhibited in the tumor microenvironment may be that the effect of an active ingredient is not exhibited in an ordinary in-vivo environment. Also, the meaning may be that there are few or no immune-related side effects caused by the activation of an active ingredient in not only the tumor microenvironment but also an ordinary in-vivo environment. Specifically, the tumor environment-sensitive cleavable polyethylene glycol-conjugated antibody for cancer treatment may undergo the release of the tumor environment-sensitive cleavable polyethylene glycol in a slightly acidic pH condition of the tumor microenvironment, so that the anticancer effect of the antibody for cancer treatment can be exhibited.

In an aspect of the present disclosure, there is provided a method for treating cancer, the method including administering a particle to a subject in need of administration thereof, the particle including: an antibody for cancer treatment; and tumor environment-sensitive cleavable polyethylene glycol.

The particle including an antibody for cancer treatment and tumor environment-sensitive cleavable polyethylene glycol or the composition containing the particle may be administered orally or parenterally, for example, through intravenous injection, subcutaneous injection, intramuscular injection, intraperitoneal injection, intrasternal injection, intratumoral injection, topical administration, intranasal administration, intrapulmonary administration, and intrarectal administration.

The appropriate dose of the particle including an antibody for cancer treatment and tumor environment-sensitive cleavable polyethylene glycol or the composition including the particle varies depending on factors, such as a formulation method, an administration method, patient's age, body weight, and sex, a pathological condition, a diet, an administration time, an administration route, an excretion rate, and response sensitivity. An ordinarily skilled practitioner can easily determine and prescribe the dose that is effective for desired treatment or prevention. According to a preferable embodiment of the present disclosure, the daily dose of the anticancer composition of the present disclosure is 0.0001-100 mg/kg.

As used herein, the term “prevention” refers to a preventive or protective treatment of a disease or a disease condition. As used herein, the term “treatment” refers to an alleviation, suppression, amelioration, or eradication of a disease condition.

In an embodiment of the present disclosure, the cancer is selected from:
(a) a blood cancer selected from the group consisting of acute lymphoblastic leukemia (ALL), acute myelogenous leukemia (AML), non-Hodgkin lymphoma (e.g., Burkitt lymphoma), B-lymphoblastic leukemia/lymphoma; B-cell chronic lymphocytic leukemia, chronic lymphocytic leukemia (CLL), chronic myelocytic leukemia (CML), follicular lymphoma, small lymphocytic lymphoma (SLL), central nervous system lymphoma, Richter's syndrome, multiple myeloma, immunoblastic large cell lymphoma, precursor B-lymphoblastic lymphoma, and anaplastic large cell lymphoma; or
(b) a solid tumor selected from the group consisting of lung cancer, pancreatic cancer, breast cancer, liver cancer, ovarian cancer, testicular cancer, kidney cancer, bladder cancer, brain cancer, cervical cancer, colon/rectal cancer, gastrointestinal cancer, skin cancer, and prostate cancer. However, the cancer is not limited thereto.

In an embodiment of the present disclosure, the antibody for cancer treatment is an immune checkpoint inhibitor antibody.

In an embodiment of the present disclosure, the composition can reduce an immune-related side effect of the immune checkpoint inhibitor antibody.

In an embodiment of the present disclosure, the immune-related side effect of the immune checkpoint inhibitor antibody is selected from the group consisting of anemia, colitis, hypothyroidism, hyperthyroidism, pneumonitis, vitiligo, pruritus, rash, autoimmune hepatitis, diarrhea, and diabetes.

The immune checkpoint inhibitor antibody has the potential to cause immune-related side effects. Side effects, such as autoimmune reactions, in the skin, gastrointestinal, endocrine, or liver due to the inhibition of immune checkpoints are known. The immune-related side effects may vary depending on the type of immune checkpoint inhibitory antibody.

In an embodiment of the present disclosure, the tumor environment-sensitive cleavable polyethylene glycol can preserve the therapeutic effects of a therapeutic drug. For example, the therapeutic drug may be an antibody, and more specifically, an immune checkpoint inhibitor antibody. The preservation of a therapeutic effect means that the binding affinity to a target, a tumor cell growth inhibitory effect, and a tumor cell killing effect are not inhibited when the antibody drug is an antibody. Alternatively, the preservation of a therapeutic effect means that the tumor environment-sensitive cleavable polyethylene glycol does not change physical and chemical structures of a drug when conjugated to or released from the drug, so that the therapeutic drug can normally exhibit therapeutic effects even after the release of the tumor environment-resistive cleavable polyethylene glycol.

Features and advantages of the present disclosure are summarized as follows.
(a) The present disclosure provides a particle including: an antibody for cancer treatment; and tumor environment-sensitive cleavable polyethylene glycol (PEG).
(b) The present disclosure provides an anticancer pharmaceutical composition containing a particle, the particle including: an antibody for cancer treatment; and tumor environment-sensitive cleavable polyethylene glycol (PEG).
(c) The use of the present disclosure can suppress side effects of the antibody for cancer treatment while maintaining therapeutic effects thereof.

### BRIEF DESCRIPTION OF THE DRAWINGS

FIG. 1 schematically shows the concepts of tumor environment-sensitive cleavable polyethylene glycol and a tumor environment-sensitive cleavable polyethylene glycol-conjugated antibody for cancer treatment of the present disclosure.

FIG. 2 shows the stepwise synthetic procedure and chemical structures of a tumor environment-sensitive cleavable polyethylene glycol-conjugated antibody for cancer treatment of the present disclosure.

FIG. 3 shows the structure of tumor environment-sensitive cleavable polyethylene glycol confirmed through ¹H NMR analysis.

FIG. 4 shows characteristics of tumor environment-sensitive cleavable polyethylene glycol released at different pH. Panel A of FIG. 4 shows the measurement results of hydrodynamic sizes of antibodies through dynamic light scattering (DLS). Panel B of FIG. 4 shows the cleavability of tumor environment-sensitive cleavable polyethylene glycol at different pH environments. Panel C of FIG. 4 shows the flow cytometry results of binding affinity of each αCD47 antibody toward CD47, expressed in mouse colon cancer line MC38 cells, according to the conjugation of tumor environment-sensitive cleavable polyethylene glycol, depending on the antibody concentration, as analyzed by flow cytometry.

FIG. 5 shows far-UV CD spectrum analysis results of αCD47 antibodies.

FIG. 6 shows the change in cancer cell phagocytosis inducing function of αCD47 antibody according to the conjugation of tumor environment-sensitive cleavable polyethylene glycol. Panel A of FIG. 6 shows representative images of flow cytometry analysis showing cancer cell phagocytosis-inducing ability of macrophages according to the antibody treatment, and panel B of FIG. 6 is a graph showing the level of phagocytosis induction (n=3).

FIG. 7 illustrates an experiment to detect the released αCD47 antibody in the tumor microenvironment. Panel A of FIG. 7 shows a schematic diagram of the experiment. Panel B of FIG. 7 shows immunofluorescence microscopic images illustrating the distribution of released αCD47 in tumor tissues.

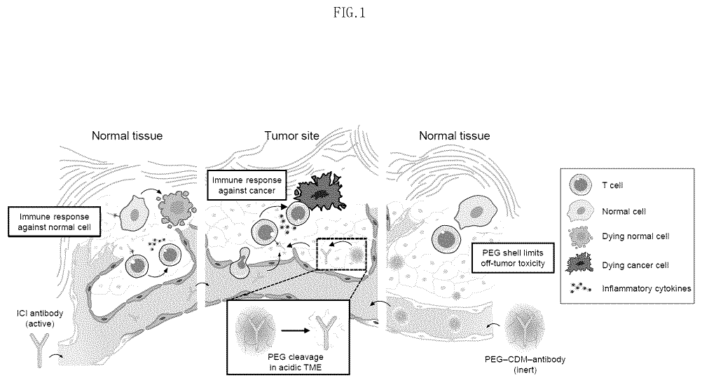

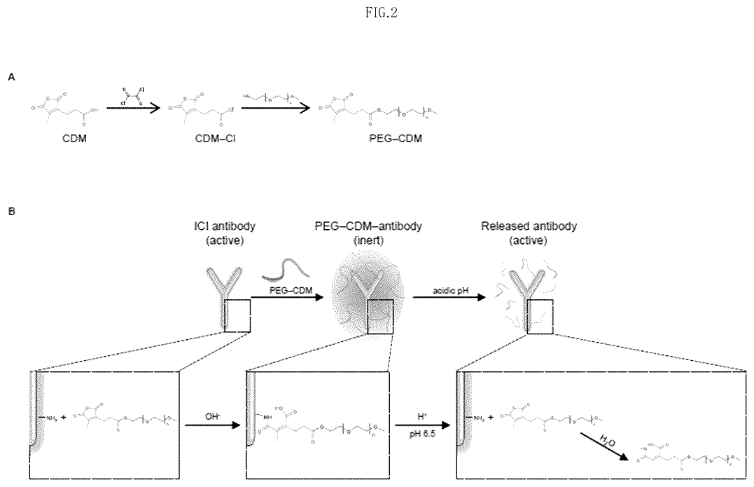

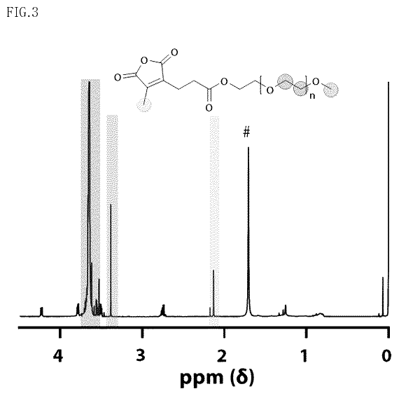

Panel A of FIG. 8 shows a schematic diagram illustrating that the phagocytosis of macrophages to erythrocytes was suppressed by the conjugation of tumor environment-sensitive cleavable polyethylene glycol, leading to the mitigation of anemia. Panel B of FIG. 8 shows the binding level between αCD47 antibody and mouse erythrocytes according to the conjugation of tumor environment-sensitive cleavable polyethylene glycol as analyzed by flow cytometry. Panel C of FIG. 8 shows confocal scanning microscopic images illustrating the change in erythrocyte phagocytosis inducing function of αCD47 antibody according to the conjugation of tumor environment-sensitive cleavable polyethylene glycol.

FIG. 9 shows the validation of side effect reducing ability of the antibody for cancer treatment according to the conjugation of tumor environment-sensitive cleavable polyethylene glycol. After an MC38 mouse tumor model was constructed, the intravenous administration of the antibody at 300 μg two times in total (Days 10 and 13) and the blood analysis four times in total (Days 9, 11, 14, and 17) were conducted. The graphs indicate changes in number of erythrocytes, number of hemoglobins, and erythrocyte volume according to the administration of αCD47 antibody.

FIG. 10 shows the validation of side effect reducing ability of cancer therapeutic antibody according to the conjugation of tumor environment-sensitive cleavable polyethylene glycol. Panel A of FIG. 10 is a schematic diagram showing that colitis syndromes could be mitigated. Panel B of FIG. 10 shows the construction of a mouse colitis model and the antibody administration manner. To construct a minimal colonic inflammatory response model, 3% DSS water was administered for three days. The antibodies αCTLA-4 and αPD-1 (ICIs) were intravenously administered at 100 μg for each. Panel C of FIG. 10 shows changes in mouse weight. Panel D of FIG. 10 shows the IL-6 level in plasma and KC level in plasma as measured by ELISA.

FIG. 11 shows the validation of side effect reducing ability of cancer therapeutic antibody according to the conjugation of tumor environment-sensitive cleavable polyethylene glycol. Panel A of FIG. 11 shows H&E staining images of colon. Panel B of FIG. 11 shows colitis scores according to the scoring system.

FIG. 12 shows the presence or absence of tissue damage in major organs by a tumor environment-sensitive cleavable polyethylene glycol-conjugated anticancer therapeutic antibody.

FIG. 13 shows the cancer therapeutic efficacy of a tumor environment-sensitive cleavable polyethylene glycol-conjugated anticancer therapeutic antibody. FIG. 13A illustrates that the removal of tumor environment-sensitive cleavable polyethylene glycol in an acidic tumor environment restores the activity of ICI antibodies. FIG. 13B shows a schematic diagram illustrating experiments to evaluate cancer therapeutic efficacy of antibodies through MC38 and B16F10 mouse tumor models. After the mouse tumor models were constructed, αCTLA-4 and αPD-1 (ICIs) were intravenously administered a total of two times at 100 μg for each time. FIG. 13C shows the survival rate of MC38 tumor constructed mice according to antibody administration. FIGS. 13D and 13E show the degree of MC38 tumor growth for each individual according to antibody administration through tumor volume and tumor weight. FIGS. 13F and 13G show the degree of B16F10 tumor growth according to antibody administration through tumor volume and tumor weight.

FIG. 14 shows the presence or absence of antibody therapeutic effect by the conjugation of tumor environment-sensitive cleavable polyethylene glycol. FIG. 14A shows the results of quantitative analysis of antibodies in plasma over time after the treatment with each antibody. FIG. 14B shows the residual and accumulation amounts of antibodies over time in the major organs and tumor. FIGS. 14C and 14D show the average radiant efficiency of each antibody over time in the major organs and tumor.

### DETAILED DESCRIPTION OF THE EXEMPLARY EMBODIMENTS

Hereinafter, the present disclosure will be described in more detail with reference to exemplary embodiments. These exemplary embodiments are provided only for the purpose of illustrating the present disclosure in more detail, and therefore, according to the purpose of the present disclosure, it would be apparent to a person skilled in the art that these exemplary embodiments are not construed to limit the scope of the present disclosure.

### EXAMPLES

### Example 1: Construction of Tumor Environment-Sensitive Cleavable Polyethylene Glycol-Conjugated Antibodies for Cancer Treatment

1-1. Preparation of Tumor Environment-Sensitive Cleavable Polyethylene Glycol (PEG-CDK)

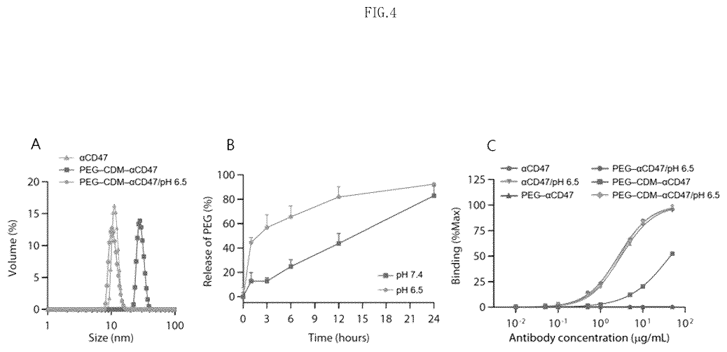

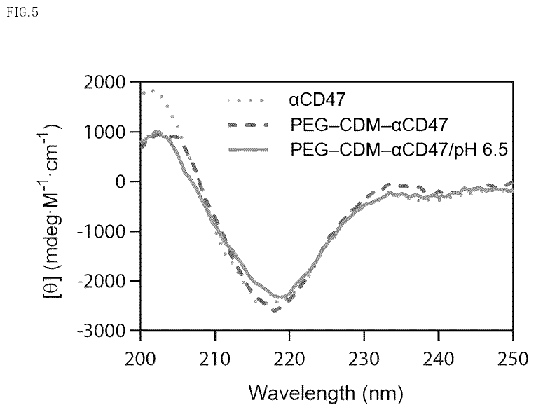

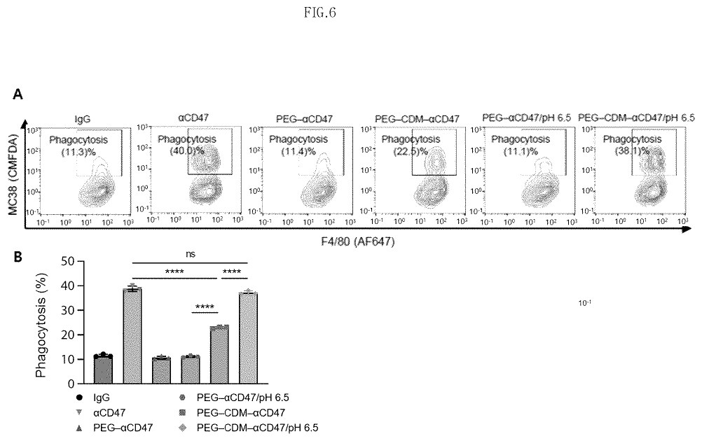

A schematic diagram of the preparation of tumor environment-sensitive cleavable polyethylene glycol is shown in FIG. 2.

Carboxy dimethylmaleic anhydride (CDM), which is released by responding to a weakly acidic environment, such as the tumor microenvironment, and oxalyl chloride are stirred in the presence of dimethyl formamide (DMF) at 0° C. for 30 minutes and then stirred at room temperature for 3 hours, thereby preparing chlorinated CDM.

After vacuum-dried for 1 day, the chlorinated CDM was stirred together with polyethylene glycol (PEG) in the presence of a pyridine catalyst at 0° C. for 30 minutes and stirred at room temperature for 24 hours. The resultant product was subjected to extraction with a saturated ammonium chloride aqueous solution, purified by diethyl ether precipitation, and then vacuum dried, thereby preparing tumor environment-sensitive cleavable polyethylene glycol (PEG-CDM).

The chemical structure of the prepared tumor environment-sensitive cleavable polyethylene glycol (PEG-CDM) was confirmed by NMR analysis.

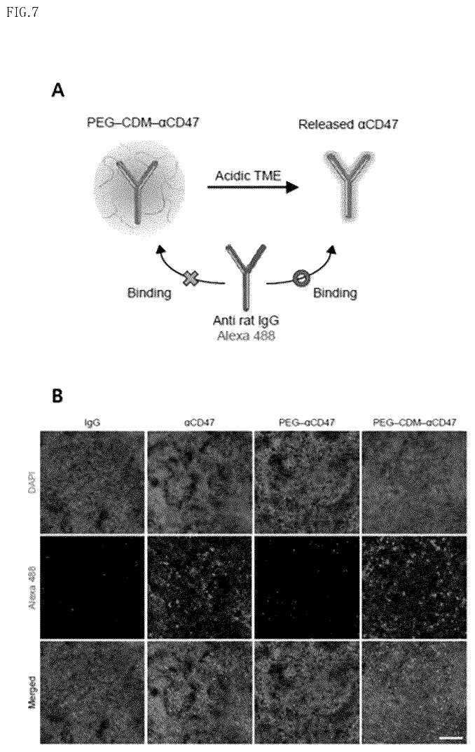

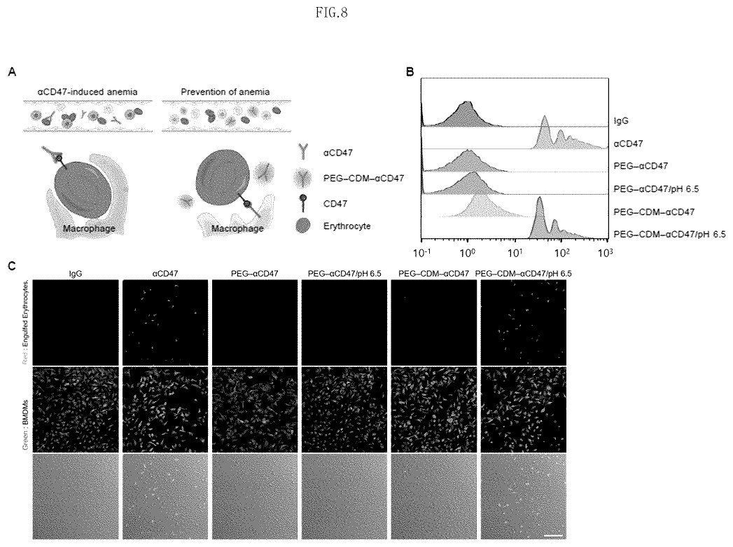

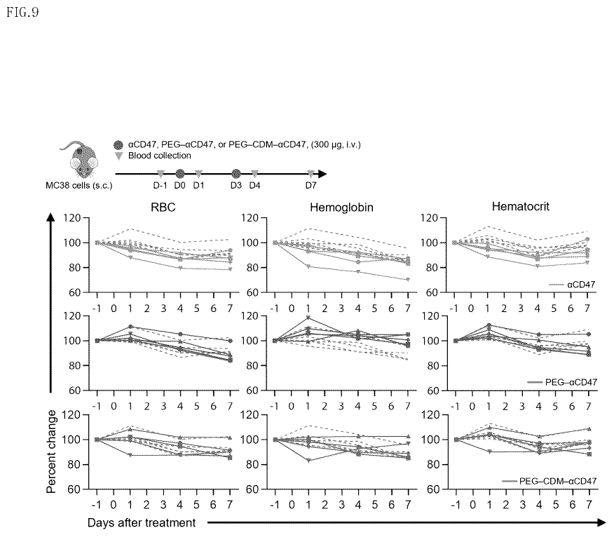

The results are shown FIG. 3.

A peak at 3.5 to 3.8 ppm(δ), a peak at 3.2 to 3.5 ppm(δ), and a peak at 2.0 to 2.5 ppm(δ) were detected as a result of ¹H-NMR measurement. The above results confirmed that PEG-CDM was successfully prepared.

1-2. Construction of Tumor Environment-Sensitive Cleavable Polyethylene Glycol-Conjugated Antibodies for Cancer Treatment

A schematic diagram of the construction of tumor environment-sensitive cleavable polyethylene glycol-conjugated antibodies for cancer treatment is shown in FIG. 2.

Antibodies (αCD47 antibody, αCTLA-4 antibody, and αPD-1 antibody), which are immune checkpoint inhibitors (ICI), among antibodies for cancer treatment, were buffer exchanged with 0.2 M borate buffer (pH 9.0), and then the tumor environment-sensitive cleavable polyethylene glycol (PEG-CDM) was added, followed by stirring at room temperature for 2 hours. Thereafter, the reaction product was purified by 0.1 M phosphate buffer (pH 7.4) to construct tumor environment-sensitive cleavable polyethylene glycol-conjugated antibodies for cancer treatment (PEG-CDM-ICI antibodies).

The conjugation proportions of antibodies for cancer treatment and tumor environment-sensitive cleavable polyethylene glycol were confirmed by TNBS analysis.

The results are shown in Table 1.

TABLE 1
The number of primary amino groups of antibody for cancer
treatment and the number of conjugated PEG molecules.

| Antibody | | Conjugated | Conjugated |
|---|---|---|---|
| (clone, | Primary | PEG-CDM² (% | PEG³ (% |
| subclass) | amino group¹ | conjugation) | conjugation) |
| Anti-mouse | 41.02 ± 1.75 | 18.15 ± 0.90 | 25.76 ± 0.59 |
| CD47 (MIAP301, | | (44.25 ± 2.19) | (62.80 ± 1.44) |
| rat IgG2a κ) | | | |
| Anti-mouse | 45.49 ± 2.36 | 19.85 ± 2.61 | 25.76 ± 1.02 |
| CTLA-4 (9D9, | | (43.64 ± 5.74) | (56.63 ± 2.24) |
| mouse IgG2b) | | | |
| Anti-mouse | 47.44 ± 1.17 | 24.32 ± 1.89 | 29.29 ± 0.59 |
| PD-1 (BP0146, | | (51.26 ± 3.98) | (61.74 ± 1.24) |
| rat IgG2a κ) | | | |

In Table 1, the primary amino group¹ indicates the number of amino groups on the surface of each antibody. The conjugated PEG-CDM² indicates the number of conjugated PEG-CDM molecules for each PEG-CDM-conjugated antibody. The conjugated PEG³ indicates the number of conjugated PEG molecules for each PEG-conjugated antibody. These results suggest that not only non-cleavable PEG but also tumor environment-sensitive cleavable polyethylene glycol can be successfully conjugated to an antibody.

### Example 2: Characterization of Particle Depending on pH Conditions

2-1. Characterization of Cleavage of Polyethylene Glycol Depending on pH Conditions

To investigate characteristics of tumor environment-sensitive cleavable polyethylene glycol (PEG-CDM) released promptly by responding to a weakly acidic environment, the tumor environment-sensitive cleavable polyethylene glycol-conjugated antibodies for cancer treatment (PEG-CDM-ICIs) each were buffer exchanged with phosphate buffer (pH 7.4 or pH 6.5), and then measured for the size change through dynamic light scattering, and some were collected at a predetermined time and the cleavage behavior of PEG was evaluated by 2,4,6-trinitrobenzene sulfonic acid (TNBS) assay.

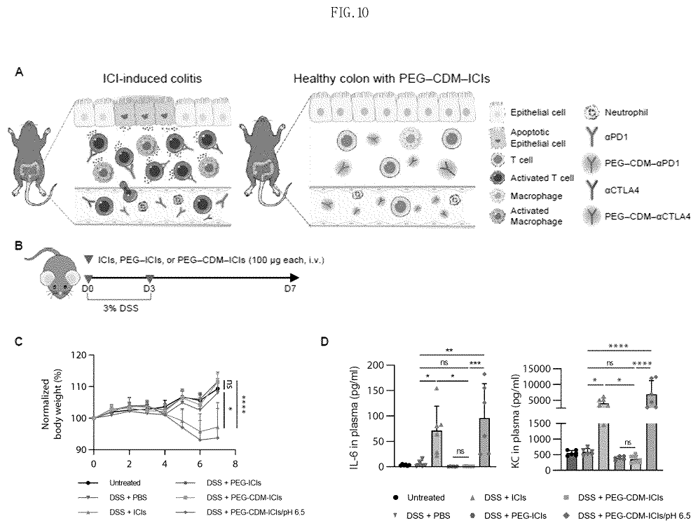

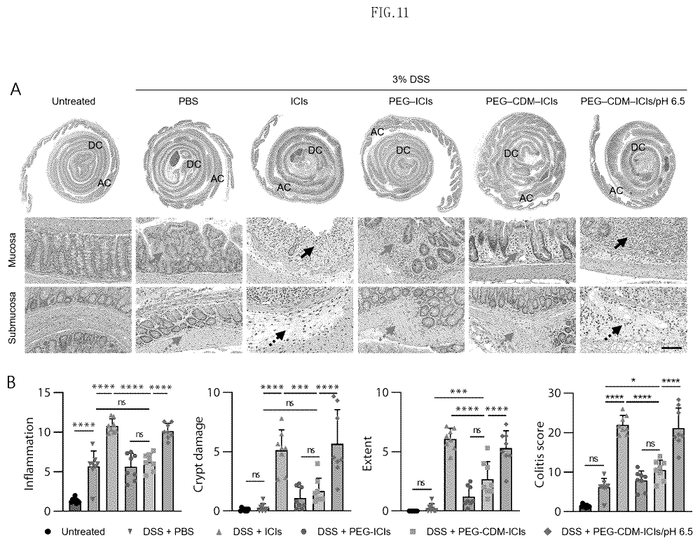

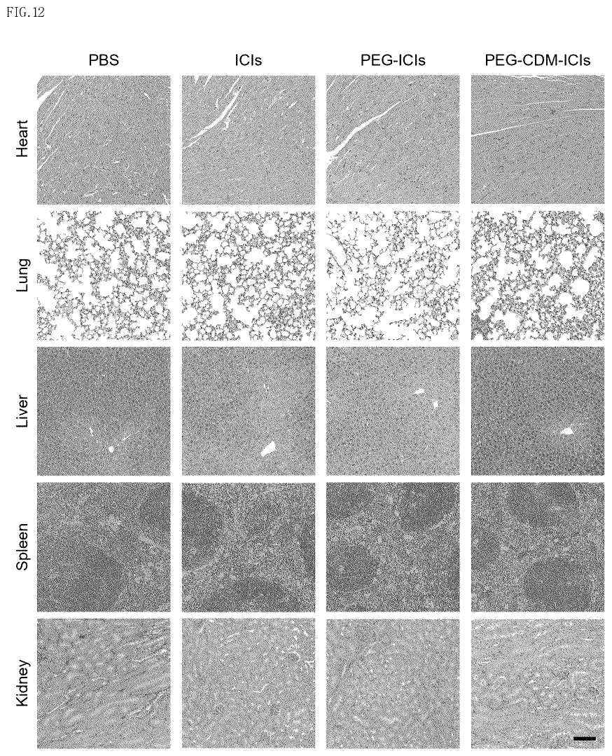

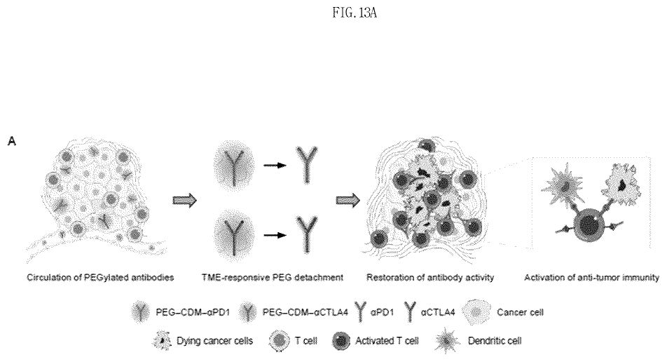

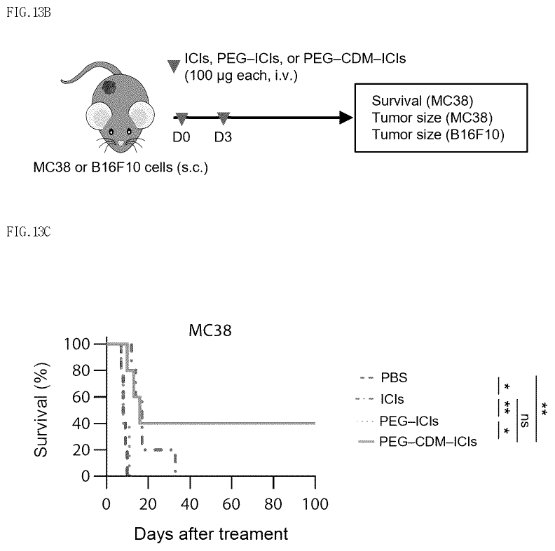

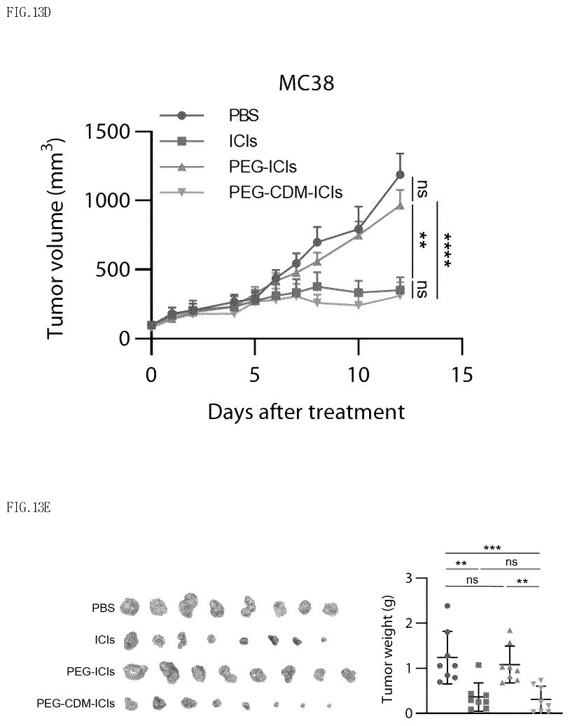

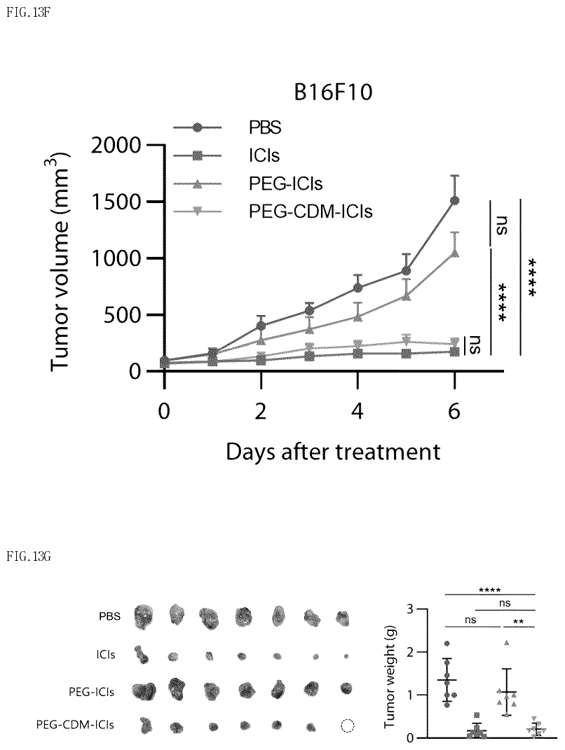

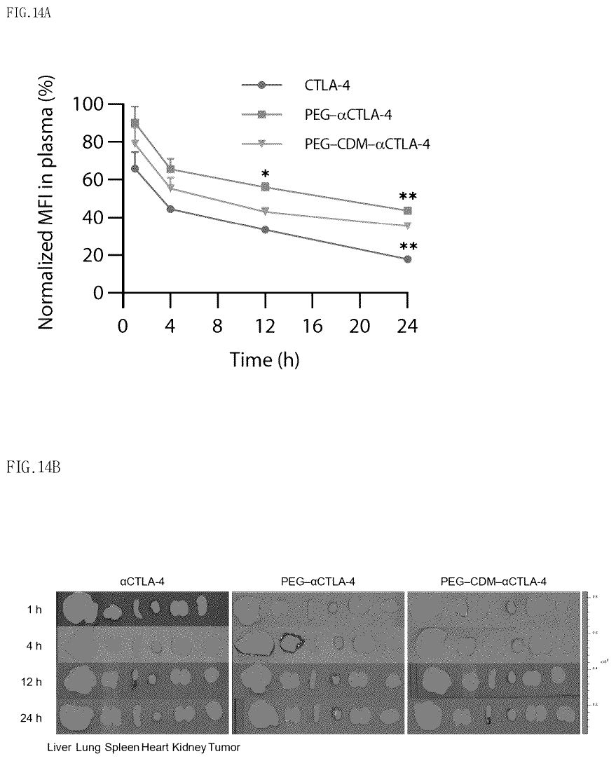

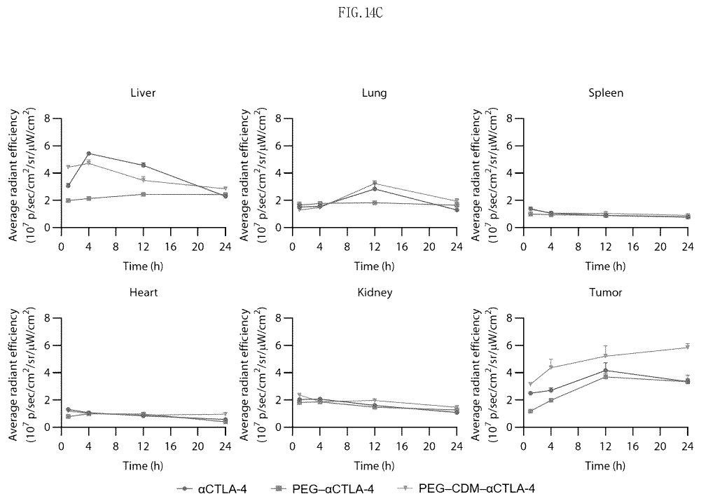

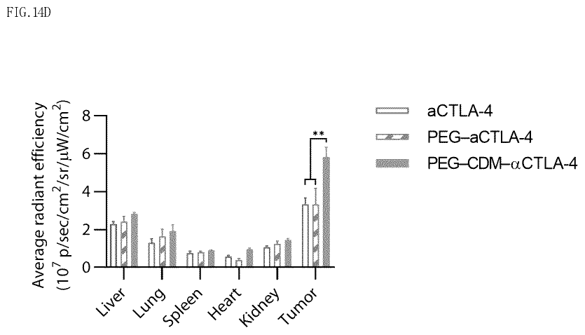

The results are shown in FIG. 4 and Table 2.

As shown in panel A of FIG. 4 and Table 2, when the tumor environment-sensitive cleavable polyethylene glycol-conjugated antibody for cancer treatment was pre-incubated in a buffer solution (pH 6.5), the tumor environment-sensitive cleavable polyethylene glycol was removed from the particle, resulting in the restoration to the original size of the antibody for cancer treatment.

TABLE 2
Comparison of particle size among αCD47 antibody,
PEG-CDM-αCD47, and PEG-CDM-αCD47 pre-incubated
in buffer solution of pH 6.5

| | αCD47 | | PEG-CDM- |
|---|---|---|---|
| | antibody | PEG-CDM-αCD47 | αCD47/pH 6.5 |
| Diameter (nm) | 11.46 ± 0.13 | 33.38 ± 3.82 | 10.83 ± 0.21 |

As shown in panel B of FIG. 4, the change in number of residual amino groups of PEG-CDM-αCD47 was quantified at each time, and these results were used to calculate the proportion of the removed tumor environment-sensitive cleavable polyethylene glycol (PEG-CDM), and as a result, the tumor environment-sensitive cleavable polyethylene glycol (PEG-CDM) conjugated to an antibody was rapidly removed in an environment of pH 6.5 rather than pH 7.4 corresponding to a physiological environment. At least half of PEG-CDM was released at pH 6.5 within 3 hours. In contrast, only 40% was dissociated from the conjugate at a physiological condition (pH 7.4) even after 12 hours.

2-2. Target-Binding Ability of Antibody for Cancer Treatment Depending on pH Conditions

The target-binding abilities of the antibody for cancer treatment (αCD47 antibody), non-cleavable polyethylene glycol-conjugated antibody for cancer treatment (PEG-αCD47), and tumor environment-sensitive cleavable polyethylene glycol-conjugated antibody for cancer treatment (PEG-CDM-αCD47) were analyzed depending on the pH conditions.

The results are shown in panel C of FIG. 4.

The binding abilities of the αCD47 antibody were equivalent even under different pH conditions, and the binding ability of PEG-αCD47 was completely lost regardless of pH conditions due to the conjugated PEG. PEG-CDM-αCD47 showed a restricted binding ability, but showed the equivalent binding ability as the original antibody at pH 6.5 since the conjugated PEG-CDM was all removed (n=3). These results suggest that the conjugation of PEG-CDM does not cause structural changes, and after PEG-CDM was released by responding to the tumor environment, the therapeutic effect of the antibody could be restored successfully.

2-3. Phagocytosis-Inducing Ability of Antibody for Cancer Treatment Depending on pH Conditions

To evaluate the intrinsic functional action of the antibody according to the conjugation of tumor environment-sensitive cleavable polyethylene glycol, flow cytometry was performed. The phagocytosis-inducing ability, which is an intrinsic function of αCD47 antibody, was investigated using MC38 cancer cell line and mouse bone marrow-derived macrophages. MC38 cancer cells stained with CMFDA were co-cultured with bone marrow-derived macrophages in the presence of each antibody at a concentration of 10 μg/mL, and the percentage of phagocytosis was investigated.

The results are shown FIG. 6.

The αCD47 antibody accelerated the phagocytosis of macrophages, but the conjugation of PEG to the antibody inhibited the binding between the antibody and the cells, thereby suppressing the induction of phagocytosis. However, when PEG-CDM-αCD47 was exposed to an environment of pH 6.5, PEG-CDM was removed and the phagocytosis-inducing ability was restored to the equivalent level to the original antibody.

2-4. Detection of Released αCD47 Antibody in Tumor Microenvironment

It was investigated whether an antibody for cancer treatment was released in the tumor microenvironment due to the removal of cleavable polyethylene glycol from the tumor environment-sensitive cleavable polyethylene glycol-conjugated antibody for cancer treatment. The antibody was intravenously administered to the MC38 mouse tumor model, and then tumor was extracted. The degree of accumulation of αCD47 in the extracted tumor was investigated through immunofluorescence.

The results are shown FIG. 7.

From the administered tumor environment-sensitive cleavable polyethylene glycol-conjugated antibody for cancer treatment (PEG-CDM-αCD47 antibody), the tumor environment-sensitive cleavable polyethylene glycol (PEG-CDM) was removed by a weakly acidic tumor tissue environment, and thus the antibody was accumulated in the tumor at the equivalent level to the original antibody for cancer treatment (αCD47 antibody).

### Example 3: Validation of Immune-Related Side Effect Reducing Ability

Anemia is a representative immune-related side effect of αCD47 antibody, and colitis is one of the representative side effects of αPD-1/αCTLA-4 antibodies. To evaluate the immune-related side effect reducing ability of the tumor environment-sensitive cleavable polyethylene glycol-conjugated antibody in an animal model, a cancer animal model and a colitis animal model were manufactured and evaluated for the degrees of induction of anemia and colitis.

3-1. Anemia

The conjugation of PEG chains to an antibody inhibits the ability of αCD47 antibody to bind to CD47 expressed in erythrocytes, results in the inhibition of phagocytosis. The degree of binding between each antibody and mouse erythrocytes was investigated through flow cytometry.

The results are shown FIG. 8.

In panel B of FIG. 8, the tumor environment-sensitive cleavable polyethylene glycol-conjugated antibody for cancer treatment (PEG-CDM-αCD47) showed a reduced binding ability to erythrocytes due to tumor environment-sensitive cleavable polyethylene glycol (PEG-CDM).

Panel C of FIG. 8 shows confocal scanning microscope images illustrating the phagocytosis induced after co-culture of mouse erythrocytes stained with pHrodo and mouse bone marrow-derived macrophages stained with CMFDA in the presence of each antibody (10 μg/ml). When the phagocytosis was induced, erythrocytes invaded into macrophages to form phagosomes, wherein the pHrodo staining exhibits fluorescence due to the acidity of the phagosomes. That is, the parts marked in red represent erythrocytes that were decomposed inside the macrophages (green) by phagocytosis. PEG-CDM-αCD47 antibody inhibited phagocytosis at the similar level to the IgG control and PEG-αCD47 antibody. However, PEG-CDM-αCD47 pre-incubated at pH 6.5 induced phagocytosis at the similar level to αCD47, and therefore, it could be confirmed that such a phagocytosis inhibitory effect disappeared when PEG-CDM-αCD47 was pre-incubated at pH 6.5. These results suggest that the targeting capacity and effector functions of medicines can be effectively controlled by PEG-CDM.

A cancer animal model was prepared by subcutaneous injection of MC38 cancer cells into mice. To evaluate anemia alleviating ability of the tumor environment-sensitive cleavable polyethylene glycol-conjugated antibody for cancer treatment (PEG-CDM-αCD47 antibody), three control groups including PBS, αCD47 antibody, and non-cleavable polyethylene glycol-conjugated antibody for cancer treatment (PEG-αCD47 antibody) were intravenously injected (two times for 4 days, 300 μg per each time). Starting the day before the first intravenous injection, the venous blood was collected four times for 8 days and an ordinary blood test was performed.

The results are shown FIG. 9.

As known, αCD47 antibody induced anemia in mice due to reductions in number of erythrocytes, number of hemoglobins, and erythrocyte volume (hematocrit). Similar to PBS, the tumor environment-sensitive cleavable polyethylene glycol-conjugated antibody for cancer treatment (PEG-CDM-αCD47 antibody) and the non-cleavable polyethylene glycol-conjugated antibody for cancer treatment (PEG-αCD47 antibody) showed normal levels in terms of the number of erythrocytes, number of hemoglobins, and erythrocyte volume (hematocrit). In other words, the conjugation of polyethylene glycol is considered to reduce or prevent the immune-related side effects of αCD47 antibody.

The reason is that αCD47 antibody, when intravenously administered, first binds to erythrocytes in the blood before reaching a cancer tissue as a target, and thus the erythrocytes are removed by macrophages to cause anemia, but the conjugation of PEG to the antibody prevents the binding between the antibody and erythrocytes, thereby alleviating anemia.

3-2. Colitis

FIG. 10 schematically shows the development of colitis, which is a representative immune-related side effect caused by an antibody for cancer treatment (A of FIG. 10). The colitis alleviating effect of the antibody for cancer treatment due to the conjugation of tumor environment-sensitive cleavable polyethylene glycol was investigated. A mouse colitis model was prepared using dextran sodium sulfate (DSS). To evaluate the colitis alleviating ability by co-administration of tumor environment-sensitive cleavable polyethylene glycol-conjugated antibodies for cancer treatment (PEG-CDM-αPD-1/αCTLA-4 antibody, PEG-CDM-ICIs), four types of antibodies including PBS, an antibody for cancer treatment (αPD-1/αCTLA-4 antibody), a non-cleavable polyethylene glycol-conjugated antibody for cancer treatment (PEG-αPD-1/αCTLA-4 antibody), and a tumor environment-sensitive cleavable polyethylene glycol-conjugated antibody for cancer treatment with PEG removed in a weakly acidic environment (PEG-CDM-αPD-1/αCTLA-4 antibody, PEG-CDM-ICIs/pH 6.5) were intravenously injected (two times for 4 days, each antibody at 100 μg for each time, a total of 200 μg). Seven days after the first injection, the colon tissue and blood were analyzed to determine the degree of inflammation (panel B of FIG. 10).

The results are shown in FIGS. 10 and 11.

As shown in panel D of FIG. 10, similar to the non-cleavable polyethylene glycol-conjugated antibody for cancer treatment, the tumor environment-sensitive cleavable polyethylene glycol-conjugated antibody for cancer treatment showed the secretion of minimal inflammatory cytokine (IL-6) and chemokine (KC) at the equivalent level to PBS. Whereas, the removal of PEG in a weakly acidic (pH 6.5) environment showed high levels of inflammatory cytokine (IL-6) and chemokine (KC) secretion, similar to the antibody for cancer treatment administration group.

The above results could be confirmed by histological analysis. As shown in panel A of FIG. 11, the antibody for cancer treatment showed epithelial damage, but the PEG-CDM-conjugated antibody showed significantly less epithelial damage, similar to the PBS-treated group. However, the group treated with the PEG-CDM-conjugated antibody for cancer treatment pre-incubated at pH 6.5 showed epithelial damage. As shown in panel B of FIG. 11, the colitis did not worsen in the PEG-CDM-conjugated antibody for cancer treatment as measured for colitis-related indicators.

The damage caused by antibody treatment in major organs was investigated. Healthy mice were administered (i.v.) with an antibody for cancer treatment (αPD-1/αCTLA-4 antibody), a non-cleavable polyethylene glycol-conjugated antibody for cancer treatment (PEG-αPD-1/αCTLA-4 antibody), a tumor environment-sensitive cleavable polyethylene glycol-conjugated antibody for cancer treatment (PEG-CDM-αPD-1/αCTLA-4 antibody, PEG-CDM-ICIs) at 100 μg for each, and 2 days after, the organs were collected and stained with hematoxylin and eosin (H&E).

The results are shown FIG. 12.

As shown in FIG. 12, the PEG-CDM-conjugated antibody for cancer treatment showed less damage to organs, similar to the PBS-treated group as evaluated for antibody treatment-causing damage in the major organs.

The above results suggest that when undergoing the removal of polyethylene glycol in weakly acidic conditions, the tumor environment-sensitive cleavable polyethylene glycol (PEG-CDM)-conjugated antibody for cancer treatment had fully recovered antibody functions, causing immune-related side effects at the equivalent level to the antibody for cancer treatment, but reduces and prevents immune-related side effects under a normal physiological condition (pH 7.4).

### Example 4: Validation of Tumor Therapeutic Effects

It was investigated whether the tumor environment-sensitive cleavable polyethylene glycol-conjugated antibody for cancer treatment had excellent cancer therapeutic efficacy compared with the non-cleavable polyethylene glycol-conjugated antibody for cancer treatment. A schematic diagram therefor is shown in FIG. 13A. A cancer animal model was prepared by subcutaneous injection of MC38 cancer cells into mice. The mice were intravenously injected with a total four types including PBS, an antibody for cancer treatment (αPD-1/αCTLA-4 antibody), a tumor environment-sensitive cleavable polyethylene glycol-conjugated antibody for cancer treatment (PEG-CDM-αPD-1/αCTLA-4 antibody), and a non-cleavable polyethylene glycol-conjugated antibody for cancer treatment (PEG-αPD-1/αCTLA-4 antibody) (two times for 4 days, each antibody at 100 μg for each time, a total of 200 μg) (FIG. 13B).

The results are shown in FIGS. 13C, 13D, 13E, 13F, and 13G.

As shown in FIG. 13C, compared with the PBS-treated control group, the group treated with the PEG-conjugated antibody for cancer treatment (PEG-ICIs) showed no significant difference in survival rate. These results suggest that the antibody lost its therapeutic activity by the non-cleavable PEG. In contrast, a high level of survival rate improving effect was confirmed in the group treated with the PEG-CDM-conjugated antibody for cancer treatment (PEG-CDM-ICIs). Notably, for a few mice, a complete therapeutic effect from tumor could be confirmed in only the group treated with the PEG-CDM-conjugated antibody for cancer treatment (PEG-CDM-ICIs).

As shown in FIGS. 13D and 13E, the tumor environment-sensitive cleavable polyethylene glycol-conjugated antibody for cancer treatment (PEG-CDM-αPD-1/αCTLA-4 antibody) showed a 75% reduction in MC38 tumor volume compared with PBS, indicating a level equivalent to the therapeutic efficacy of the antibody for cancer treatment (αPD-1/αCTLA-4 antibody) group. However, the non-cleavable polyethylene glycol-conjugated antibody for cancer treatment (PEG-αPD-1/αCTLA-4 antibody) never inhibited the tumor growth at the equivalent level to PBS. The above results suggest that the tumor environment-sensitive cleavable polyethylene glycol-conjugated antibody for cancer treatment undergo the tumor tissue-specific removal of polyethylene glycol and thus has the equivalent therapeutic efficacy to existing antibodies.

As shown in FIGS. 13F and 13G, the tumor environment-sensitive cleavable polyethylene glycol-conjugated antibody for cancer treatment (PEG-CDM-ICIs) maintained low levels of B16F10 tumor volume and weight similar to the group treated with the antibody for cancer treatment (ICIs) and thus can retain excellent cancer therapeutic effects even with the conjugation of tumor-environment-sensitive cleavable polyethylene glycol.

To re-confirm whether the conjugation of PEG-CDM affected the anti-tumor efficacy of an antibody, the antibody activity according to the conjugation and release of PEG-CDM was investigated.

The results are shown in FIGS. 14A, 14B, 14C, and 14D.

As shown in FIG. 14A, compared with the αCTLA-4 antibody, the PEG-αCTLA-4 and PEG-CDM-αCTLA-4 antibodies showed prolonged circulation, which might be attributed to the suppressed off-tumor binding.

As shown in FIGS. 14B, 14C, and 14D, only the PEG-CDM-αCTLA-4 antibody showed enhanced tumor accumulation as evaluated through ex-vivo quantitative analysis of the tumor and major organs for 24 hours.

What is claimed is:

1. A particle comprising: an antibody for cancer treatment; and tumor environment-sensitive cleavable polyethylene glycol (PEG).

2. The particle of claim 1, wherein the tumor environment-sensitive cleavable polyethylene glycol is a linkage of a pH-sensitive molecule and polyethylene glycol.

3. The particle of claim 2, wherein the pH-sensitive molecule is an acid labile linker or a dimethyl maleic anhydride derivative.

4. The particle of claim 3, wherein the dimethyl maleic anhydride derivative is carboxylated dimethyl maleic anhydride (CDM).

5. The particle of claim 1, wherein the tumor environment-sensitive cleavable polyethylene glycol is conjugated to the antibody for cancer treatment.

6. The particle of claim 5, wherein the tumor environment-sensitive cleavable polyethylene glycol is bound to an amino group on the surface of the antibody for cancer treatment.

7. The particle of claim 5, wherein the conjugation of the antibody for cancer treatment and the tumor environment-sensitive cleavable polyethylene glycol is dissociated at pH 5.0 to 7.0.

8. The particle of claim 1, wherein the antibody for cancer treatment is an immune checkpoint inhibitor (ICI) antibody.

9. The particle of claim 8, wherein the immune checkpoint inhibitor antibody is an antibody or an antigen-binding fragment thereof that targets at least one selected from the group consisting of CD47, PD-1, PD-L1, CTLA-4, B7-1, B7-2, LAG3, KIR, 4-1BB, and TIM-3.

10. The particle of claim 1, wherein the particle has a size of 10 nm to 200 nm.

11. A method for treating cancer, the method comprising administering a particle to a subject in need of administration thereof, the particle comprising: an antibody for cancer treatment; and tumor environment-sensitive cleavable polyethylene glycol (PEG).

12. The method of claim 11, wherein the cancer is selected from:
(a) a blood cancer selected from the group consisting of acute lymphoblastic leukemia (ALL), acute myelogenous leukemia (AML), non-Hodgkin lymphoma, B-lymphoblastic leukemia/lymphoma, B-cell chronic lymphocytic leukemia, chronic lymphocytic leukemia (CLL), chronic myelocytic leukemia (CML), follicular lymphoma, small lymphocytic lymphoma (SLL), central nervous system lymphoma, Richter's syndrome, multiple myeloma, immunoblastic large cell lymphoma, precursor B-lymphoblastic lymphoma, and anaplastic large cell lymphoma; or
(b) a solid tumor selected from the group consisting of lung cancer, pancreatic cancer, breast cancer, liver cancer, ovarian cancer, testicular cancer, kidney cancer, bladder cancer, brain cancer, cervical cancer, colon/rectal cancer, gastrointestinal cancer, skin cancer, and prostate cancer.

13. The method of claim 11, wherein the antibody for cancer treatment is an immune checkpoint inhibitor antibody.

14. The method of claim 13, wherein the method reduces an immune-related side effect of the immune checkpoint inhibitor antibody.

15. The method of claim 14, wherein the immune-related side effect of the immune checkpoint inhibitor antibody is selected from the group consisting of anemia, colitis, hypothyroidism, hyperthyroidism, pneumonitis, vitiligo, pruritus, rash, autoimmune hepatitis, diarrhea, and diabetes.
